(19) United States
(12) Patent Application Publication
(10) Pub. No.: US 2007/0243221 A1
(43) Pub. Date: Oct. 18, 2007

(54) ANTI-WRINKLE COMPOSITION

(75) Inventors: Alexandre Cavezza, Pavillon Sous Bois (FR); Philippe Breton, Noisy Le Roi (FR); Maria Dalko, Gif S/Yvette (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/733,786

(22) Filed: Apr. 11, 2007

Related U.S. Application Data
(60) Provisional application No. 60/792,327, filed on Apr. 17, 2006.

(30) Foreign Application Priority Data
Apr. 11, 2006 (FR) 06 51315

(51) Int. Cl. A61K 8/49; C07D 211/56; A61K 31/445

(52) U.S. Cl. 424/401; 514/317; 546/223

(57) ABSTRACT

The present invention relates to the cosmetic use of at least one 4-aminopiperidine compound of formula (I) 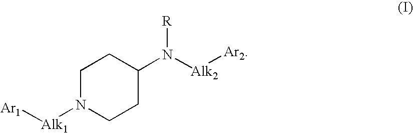 in which:
Alk₁ and Alk₂ denote a C₁-C₁₀ alkylene radical;
Ar₁ denotes a phenyl group optionally substituted with one or more radicals, which may be identical or different, chosen from —F, —CF₃, —R₁, —OR₁, —NR₁R₂;
Ar₂ denotes a phenyl group optionally substituted with one or more radicals, which may be identical or different, chosen from —F, —CF₃, —NR₁R₂;
R denotes a hydrogen atom or a C₁-C₁₀ alkyl radical;
   R₁ and R₂ denoting a C₁-C₇ alkyl radical; and the salts, optical isomers and solvates thereof, as an agent for combating wrinkles, especially expression wrinkles, and/or for decontracting the skin and/or relaxing the features. The invention also relates to a cosmetic composition containing such a compound and to the corresponding novel compounds.

### REFERENCE TO PRIOR APPLICATIONS

This application claims priority to U.S. provisional application 60/792,327 filed Apr. 17, 2006, and to French patent application 0651315 filed Apr. 11, 2006, both incorporated herein by reference.

### FIELD OF THE INVENTION

The present invention relates to the use of 4-aminopiperidine compounds as anti-wrinkle agents, and also to a composition containing such compounds, which is preferably intended to be applied to human skin. The invention also relates to novel 4-aminopiperidine compounds, and to processes for preparing 4-aminopiperidine compounds.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Women, and even men, currently have a tendency to wish to look youthful for as long as possible and consequently seek to fade out the age marks on the skin, which are reflected in particular by wrinkles and fine lines. In this respect, advertising and the fashion world report about products intended to keep the skin radiant and wrinkle-free for as long as possible, which are signs of youthful skin, all the more so since the physical appearance acts on the psyche and/or on the morale.

Hitherto, wrinkles and fine lines were treated using cosmetic products containing active agents acting on the skin, for example by improving its cell renewal or alternatively by promoting the synthesis, or by preventing the degradation, of the elastic fibres thereof of which skin tissue is composed.

Although these treatments make it possible to act on the wrinkles and fine lines caused by chronological or intrinsic ageing, and also on those caused by photoageing, they have no effect on expression wrinkles, which require an intervention on the muscular contractile component (via muscle-relaxing agents) or dermal contractile component (via dermo-decontracting agents) of wrinkles.

Specifically, expression wrinkles are the result of mechanisms different from those that generate the wrinkles caused by ageing.

Specifically, they are produced due to the effect of the strain exerted on the skin by the skin muscles that allow facial expressions. Depending on the shape of the face, the frequency of facial expressions and possible tics, they may appear even from childhood. Age, and also certain environmental factors such as exposure to sunlight, do not play a part in generating them, but may make them deeper and permanent.

Expression wrinkles are characterized by the presence of grooves around the orifices formed by the nose (nasal grooves), the mouth (perioral wrinkles and “sour-face” wrinkles) and the eyes (crow's-feet wrinkles), around which are the skin muscles, and also between the eyebrows (glabella wrinkles or lion wrinkles) and on the forehead.

Hitherto, the only means commonly used for acting on expression wrinkles is botulinum toxin, which is especially injected into the wrinkles of the glabella which are wrinkles between the eyebrows (see J. D. Carruters et al., J. Dermatol. Surg. Oncol., 1992, 18, pp. 17-21).

Various compounds capable of affording an anti-wrinkle effect when they are applied topically to the skin have been proposed, thus making it possible to act on expression wrinkles via another route.

However, there is still a need for other effective compounds for smoothing or fading out wrinkles, in particular expression wrinkles.

### SUMMARY OF THE INVENTION

The inventors have now discovered, surprisingly, that certain 4-aminopiperidine compounds satisfy this need.

One subject of the present invention is thus the use of at least one 4-aminopiperidine compound of formula (I): 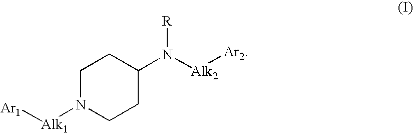
in which:
Alk₁ and Alk₂ denote, independently of each other, a linear saturated C₁-C₁₀ or unsaturated C₂-C₁₀ or branched saturated or unsaturated C₃-C₁₀ alkylene radical (divalent radical);
Ar₁ denotes a phenyl group optionally substituted with one or more radicals, which may be identical or different, chosen from —F, —CF₃, —R₁, —OR₁, —NR₁R₂;
Ar₂ denotes a phenyl group optionally substituted with one or more radicals, which may be identical or different, chosen from —F, —CF₃, —NR₁R₂;
R denotes a hydrogen atom or a saturated or unsaturated, linear C₁-C₁₀ or branched C₃-C₁₀ alkyl radical, optionally substituted with a group chosen from —Ar₁, —OR₁, —NR₁R₂;
   R₁ and R₂ denoting, independently of each other, a saturated, linear C₁-C₇, or unsaturated C₂-C₇ or saturated or unsaturated branched or cyclic C₃-C₇ alkyl radical;
   and the salts, optical isomers and solvates thereof,
   as an agent for combating wrinkles, especially expression wrinkles, and/or for decontracting the skin and/or relaxing the features.

A subject of the invention is also a cosmetic process for treating the skin, especially wrinkled skin, in particular the skin of the face and/or the forehead, comprising the topical application to the skin of a composition comprising, in a physiologically acceptable medium, at least one 4-aminopiperidine compound of formula (I) as defined above.

Some of the compounds of formula (I) are known:
1-(2-phenylethyl)-4-(N-benzyl)aminopiperidine (compound 2) is a known compound having the reference CAS 733674-39-0;
1-N-benzylamino-4-benzylpiperidine is especially described as a synthetic intermediate in patent application WO 2005/005395 (page 61, example A-2);
1-(2-phenylethyl)-4-[N-(2-phenylethyl)]aminopiperidine (compound 4) is described in patent application HU 157777325;
1-(2-phenylethyl)-4-[N-(1-phenylethyl)]aminopiperidine (compound 5) is a compound sold by the company Chembridge under the reference 5454694;
1-benzyl-4-[N-(1-phenylethyl)]aminopiperidine (compound 6) is described in the publication by Corruble A. et al. “Structure-selectivity relationship in alkyllithium-aldehyde condensation using 3-aminopyrrolidine lithium amides as chiral auxiliaries”, J. Organic. Chem. 1998, 63, 8266-8275 (compound 19b′). Patent application WO 02/083 641 describes amino-aza-cyclohexane compounds (in particular the compounds of Examples 10, 11 and 19) in pharmaceutical compositions for treating malaria.

A subject of the invention is also a composition comprising, in a physiologically acceptable medium, at least one 4-aminopiperidine compound of formula (II): 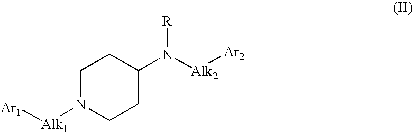
in which:
R denotes a hydrogen atom, and
Alk₁ denotes a saturated or unsaturated, linear C₂-C₁₀ or branched C₃-C₁₀ alkylene radical (divalent radical); and
Alk₂ denotes a saturated, linear C₁-C₁₀ or unsaturated C₂-C₁₀ or saturated or unsaturated branched C₃-C₁₀ alkylene radical (divalent radical); or
R denotes a saturated or unsaturated, linear C₁-C₁₀ or branched C₃-C₁₀ alkyl radical, optionally substituted with a group chosen from —Ar₁, —OR₁, —NR₁R₂; and
Alk₁ and Alk₂ denote, independently of each other, a saturated, linear C₁-C₁₀ or unsaturated C₂-C₁₀ or saturated or unsaturated branched C₃-C₁₀ alkylene radical (divalent radical);
Ar₁ denotes a phenyl group optionally substituted with one or more radicals, which may be identical or different, chosen from —F, —CF₃, —R₁, —OR₁, —NR₁R₂;
Ar₂ denotes a phenyl group optionally substituted with one or more radicals, which may be identical or different, chosen from —F, —CF₃, —NR₁R₂;
   R₁ and R₂ denoting, independently of each other, a saturated, linear C₁-C₇ or unsaturated C₂-C₇ or saturated or unsaturated branched or cyclic C₃-C₇ alkyl radical;
   and the salts, optical isomers and solvates thereof.

A subject of the invention is also novel 4-aminopiperidine compounds of formula (III): 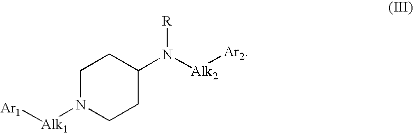
in which:
Alk₁ denotes a saturated or unsaturated, linear C₂-C₁₀ or branched C₃-C₁₀ alkylene radical (divalent radical);
Alk₂ denotes a saturated or unsaturated, linear or branched C₃-C₁₀ alkylene radical (divalent radical);
Ar₁ denotes a phenyl group optionally substituted with one or more radicals, which may be identical or different, chosen from —F, —CF₃, —R₁, —OR₁, —NR₁R₂;
Ar₂ denotes a phenyl group optionally substituted with one or more radicals, which may be identical or different, chosen from —F, —CF₃, —NR₁R₂;
R denotes a hydrogen atom or a saturated or unsaturated, linear C₁-C₁₀ or branched C₃-C₁₀ alkyl radical, optionally substituted with a group chosen from —Ar₁, —OR₁, —NR₁R₂;
   R₁ and R₂ denoting, independently of each other, a saturated or unsaturated, linear C₁-C₇ or branched or cyclic C₃-C₇ alkyl radical;
   and the salts, optical isomers and solvates thereof.

In formulae (I), (II) and (III), the alkyl groups may preferably be chosen, depending on the case, from the following groups: methyl, ethyl, n-propyl, isopropyl, n-butyl, isobutyl, tert-butyl, pentyl, hexyl, heptyl, octyl, nonyl, decyl. Similarly, the divalent alkylene groups may be chosen from methylene, ethylene, n-propylene, isopropylene, n-butylene, isobutylene, tert-butylene, pentylene, hexylene, heptylene, octylene, nonylene and decylene radicals.

For the compounds of formula (I) those having the following meanings are preferred:
Alk₁ and Alk₂ denote, independently of each other, a saturated, linear C₁-C₁₀ or branched C₃-C₁₀ alkylene radical;
Ar₁ denotes a phenyl group optionally substituted with one or more radicals, which may be identical or different, chosen from —R₁, —OR₁;
Ar₂ denotes a phenyl group optionally substituted with one or more radicals —CF₃;
R denotes a hydrogen atom or a saturated, linear C₁-C₄ or branched C₃-C₄ alkyl radical, optionally substituted with a group chosen from —Ar₁, —OR₁, —NR₁R₂;
   R₁ and R₂ denoting, independently of each other, a saturated, linear C₁-C₄ alkyl radical.

Preferentially, compounds of formula (I) are used for which:
Alk₁ and Alk₂ denote, independently of each other, a saturated, linear C₁-C₄ or branched C₃-C₄ alkylene radical;
Ar₁ and Ar₂ denote a phenyl group;
R denotes a hydrogen atom or a saturated, linear C₁-C₄ or branched C₃-C₄ alkyl radical, optionally substituted with a group chosen from —Ar₁, —OR₁, —NR₁R₂;
   R₁ and R₂ denoting, independently of each other, a saturated, linear C₁-C₄ alkyl radical.

More preferentially, compounds of formula (I) are used for which:
Alk₁ and Alk₂ denote, independently of each other, a saturated, linear C₁-C₄ or branched C₃-C₄ alkylene radical;
Ar₁ and Ar₂ denote a phenyl group;
R denotes a hydrogen atom or a saturated, linear C₁-C₄ or branched C₃-C₄ alkyl radical, optionally substituted with a group chosen from a phenyl radical, —NR₁R₂ and, preferably, a hydrogen atom.

For the compounds of formula (II), those having the following meanings are preferred:
R denotes a hydrogen atom, and
Alk₁ denotes a saturated, linear C₂-C₁₀ or branched C₃-C₁₀ alkylene radical; and
Alk₂ denotes a saturated, linear C₁-C₁₀ or branched C₃-C₁₀ alkylene radical; or
R denotes a saturated, linear C₁-C₄ or branched C₃-C₄ alkyl radical, optionally substituted with a group chosen from —Ar₁, —OR₁, —NR₁R₂; and
Alk₁ and Alk₂ denote, independently of each other, a saturated, linear C₁-C₁₀ or branched C₃-C₁₀ alkylene radical (especially such as those described above when R═H);
Ar₁ denotes a phenyl group optionally substituted with one or more radicals, which may be identical or different, chosen from —R₁, —OR₁;
Ar₂ denotes a phenyl group optionally substituted with one or more —CF₃ radicals;
   R₁ and R₂ denoting, independently of each other, a saturated, linear C₁-C₄ alkyl radical.

Preferentially, compounds of formula (II) are used for which:
R denotes a hydrogen atom, and
Alk₁ denotes a saturated, linear C₂-C₄ or branched C₃-C₄ alkylene radical; and
Alk₂ denotes a saturated, linear C₁-C₄ or branched C₃-C₄ alkylene radical; or
R denotes a saturated, linear C₁-C₄ or branched C₃-C₄ alkyl radical, optionally substituted with a group chosen from —Ar₁, —OR₁, —NR₁R₂; and
Alk₁ and Alk₂ denote, independently of each other, a saturated, linear C₁-C₄ or branched C₃-C₄ alkylene radical (especially such as those described above when R═H);
Ar₁ and Ar₂ denote a phenyl group;
   R₁ and R₂ denoting, independently of each other, a saturated, linear C₁-C₄ alkyl radical.

More preferentially, compounds of formula (II) are used for which:
R denotes a hydrogen atom, and
Alk₁ denotes a saturated, linear C₂-C₁₀ or branched C₃-C₁₀ alkylene radical; and
Alk₂ denotes a saturated, linear C₁-C₁₀ or branched C₃-C₁₀ alkylene radical; or
R denotes a saturated, linear C₁-C₄ or branched C₃-C₄ alkyl radical, optionally substituted with a group chosen from a phenyl radical, —NR₁R₂, and
Alk₁ and Alk₂ denote, independently of each other, a saturated, linear C₁-C₄ or branched C₃-C₄ alkylene radical (especially such as those described above when R═H);
Ar₁ and Ar₂ denote a phenyl group;
   R₁ and R₂ denoting, independently of each other, a saturated, linear C₃-C₄ alkyl radical.

More preferably, compounds of formula (II) are used for which:
R denotes a hydrogen atom,
Alk₁ denotes a saturated, linear C₂-C₁₀ or branched C₃-C₁₀ alkylene radical;
Alk₂ denotes a saturated, linear C₁-C₁₀ or branched C₃-C₁₀ alkylene radical;
Ar₁ and Ar₂ denote a phenyl group;
   R₁ and R₂ denoting, independently of each other, a saturated, linear C₁-C₄ alkyl radical.

For the compounds of formula (II), those having the following meanings are preferred:
R denotes a hydrogen atom, and
Alk₁ denotes a saturated, linear C₂-C₁₀ or branched C₃-C₁₀ alkylene radical; and
Alk₂ denotes a saturated, linear or branched C₃-C₁₀ alkylene radical; or
R denotes a saturated, linear C₁-C₄ or branched C₃-C₄ alkyl radical, optionally substituted with a group chosen from —Ar₁, —OR₁, —NR₁R₂; and
Alk₁ and Alk₂ denote, independently of each other, a saturated, linear C₁-C₁₀ or branched C₃-C₁₀ alkylene radical (especially such as those described above when R═H);
Ar₁ denotes a phenyl group optionally substituted with one or more radicals, which may be identical or different, chosen from —R₁, —OR₁;
Ar₂ denotes a phenyl group optionally substituted with one or more —CF₃ radicals;
   R₁ and R₂ denoting, independently of each other, a saturated, linear C₁-C₄ alkyl radical.

Preferentially, compounds of formula (III) are used for which:
R denotes a hydrogen atom, and
Alk₁ denotes a saturated, linear C₂-C₄ or branched C₃-C₄ alkylene radical; and
Alk₂ denotes a saturated, linear or branched C₃-C₄ alkylene radical; or
R denotes a saturated, linear C₁-C₄ or branched C₃-C₄ alkyl radical, optionally substituted with a group chosen from —Ar₁, —OR₁, —NR₁R₂; and
Alk₁ and Alk₂ denote, independently of each other, a saturated, linear C₁-C₄ or branched C₃-C₄ alkylene radical (especially such as those described above when R═H);
Ar₁ and Ar₂ denote a phenyl group;
   R₁ and R₂ denoting, independently of each other, a saturated, linear C₁-C₄ alkyl radical.

More preferentially, compounds of formula (III) are used for which:
R denotes a hydrogen atom, and
Alk₁ denotes a saturated, linear C₂-C₄ or branched C₃-C₄ alkylene radical; and
Alk₂ denotes a saturated, linear or branched C₃-C₄ alkylene radical; or
R denotes a saturated, linear C₁-C₄ or branched C₃-C₄ alkyl radical, optionally substituted with a group chosen from a phenyl radical, —NR₁R₂, and
Alk₁ and Alk₂ denote, independently of each other, a saturated, linear C₁-C₄ or branched C₃-C₄ alkylene radical (especially such as those described above when R═H);
Ar₁ and Ar₂ denote a phenyl group;
   R₁ and R₂ denoting, independently of each other, a saturated, linear C₁-C₄ alkyl radical.

More preferably, compounds of formula (III) are used for which:
R denotes a hydrogen atom, and
Alk₁ denotes a saturated, linear C₂-C₄ or branched C₃-C₄ alkylene radical; and
Alk₂ denotes a saturated, linear or branched C₃-C₄ alkylene radical;
Ar₁ and Ar₂ denote a phenyl group;
   R₁ and R₂ denoting, independently of each other, a saturated, linear C₁-C₄ alkyl radical.

The preferred salts of the compounds described in the present invention comprise conventional non-toxic salts of the compounds, such as those formed from organic or mineral acids. Examples that may be mentioned include the salts of mineral acids, such as sulfuric acid, hydrochloric acid, hydrobromic acid, hydriodic acid, phosphoric acid or boric acid. Mention may also be made of the salts of organic acids, which may include one or more carboxylic, sulfonic or phosphonic acid groups. They may be linear, branched or cyclic aliphatic acids, or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

The highly preferred salts are those obtained from hydrochloric acid, sulfuric acid, acetic acid, tartaric acid or citric acid.

The acceptable solvates of the compounds described in the present invention comprise conventional solvates such as those formed during the final step of preparation of the compounds due to the presence of solvents. Examples that may be mentioned include solvates due to the presence of water or of linear or branched alcohols, for instance ethanol or isopropanol.

Among the preferred compounds of formula (I) that may be mentioned are:

| Compound | NAME | Ar₁ | Alk₁ | Alk₂ | Ar₂ | R |
|---|---|---|---|---|---|---|
| 1 | 1-(2-phenylethyl)-4-[N-(3- | Ph | CH₂CH₂ | CH₂CH₂CH₂ | Ph | H |
| | phenylpropyl)]amino- | | | | | |
| | piperidine | | | | | |
| 2 | 1-(2-phenylethyl)-4-(N- | Ph | CH₂CH₂ | CH₂ | Ph | H |
| | benzyl)aminopiperidine | | | | | |
| | (known compound) | | | | | |
| 3 | 1-(2-phenylethyl)-4-[N-(3,5- | Ph | CH₂CH₂ | CH₂ | (3,5)-CF₃-Ph | H |
| | bistrifluoromethyl)benzyl]- | | | | | |
| | aminopiperidine | | | | | |
| 4 | 1-(2-phenylethyl)-4-[N-(2- | Ph | CH₂CH₂ | CH₂CH₂ | Ph | H |
| | phenylethyl)]amino- | | | | | |
| | piperidine | | | | | |
| | (known compound) | | | | | |
| 5 | 1-(2-phenylethyl)-4-[N-(1- | Ph | CH₂CH₂ | CHCH₃ | Ph | H |
| | phenylethyl)]amino- | | | | | |
| | piperidine | | | | | |
| | (known compound) | | | | | |
| 6 | 1-benzyl-4-[N-(1-phenyl- | Ph | CH₂ | CHCH₃ | Ph | H |
| | ethyl)]aminopiperidine | | | | | |
| | (known compound) | | | | | |
| 7 | 1-[2-(3,4-dimethoxy)- | (3,4)-OMe-Ph | CH₂CH₂ | CH₂CH₂CH₂ | Ph | H |
| | phenylethyl]-4-[N-(3- | | | | | |
| | phenylpropyl)]amino- | | | | | |
| | piperidine | | | | | |
| 8 | 1-(3-phenylpropyl)-4-[N-(3- | Ph | CH₂CH₂CH₂ | CH₂CH₂CH₂ | Ph | H |
| | phenylpropyl)]amino- | | | | | |
| | piperidine | | | | | |
| 9 | 1-(2-phenylethyl)-4-[N-(4- | Ph | CH₂CH₂ | CH₂CH₂CH₂CH₂ | Ph | H |
| | phenylbutyl)]amino- | | | | | |
| | piperidine | | | | | |
| 10 | 1-(2-phenylethyl)-4-[N-(1- | Ph | CH₂CH₂ | CHCH₂CH₃ | Ph | H |
| | phenylpropyl)]amino- | | | | | |
| | piperidine | | | | | |
| 11 | 1-[2-(4-tert-butyl)phenyl- | 4-tBu-Ph | CH₂CH₂ | CH₂CH₂CH₂ | Ph | H |
| | ethyl]-4-[N-(3-phenyl- | | | | | |
| | propyl)]aminopiperidine | | | | | |
| 12 | 1-(2-phenylethyl)-4-[N- | Ph | CH₂CH₂ | CHCH₃ (S) | Ph | H |
| | (1S)-1-phenylethyl]amino- | | | | | |
| | piperidine | | | | | |
| 13 | 1-[2-(4-tert-butyl)phenyl- | 4-tBu-Ph | CH₂CH₂ | CHCH₃ | Ph | H |
| | ethyl]-4-[N-(1-phenyl- | | | | | |
| | ethyl)]aminopiperidine | | | | | |
| 14 | 1-(4-phenylbutyl)-4-[N- | Ph | CH₂CH₂CH₂CH₂ | CH₂CH₂CH₂ | Ph | H |
| | (3-phenylpropyl)]amino- | | | | | |
| | piperidine | | | | | |
| 15 | 1-(3-phenylpropyl)-4-[N- | Ph | CH₂CH₂CH₂ | CHCH₂CH₃ | Ph | H |
| | (1-phenylpropyl)]amino- | | | | | |
| | piperidine | | | | | |
| 16 | 1-(3-phenylpropyl)-4-[N- | Ph | CH₂CH₂CH₂ | CHCH₃ (S) | Ph | H |
| | (1S)-1-phenylethyl]amino- | | | | | |
| | piperidine | | | | | |
| 17 | 1-(2-phenylethyl)-4-[N- | Ph | CH₂CH₂ | CH₂CH₂CH₂ | Ph | Me |
| | methyl-N-(3-phenyl- | | | | | |
| | propyl)]aminopiperidine | | | | | |
| 18 | 1-(2-phenylethyl)-4-[N,N- | Ph | CH₂CH₂ | CH₂CH₂ | Ph | CH₂CH₂Ph |
| | di(2-phenylethyl)]amino- | | | | | |
| | piperidine | | | | | |
| 19 | 1-(2-phenylethyl)-4-[N- | Ph | CH₂CH₂ | CHCH₃ | Ph | Et |
| | ethyl-N-(1-phenylethyl)]- | | | | | |
| | aminopiperidine | | | | | |
| 20 | 1-(2-phenylethyl)-4-[N-(2- | Ph | CH₂CH₂ | CHCH₃ | Ph | CH₂CH₂CH₂Ph |
| | phenylpropyl)-N-(1-phenyl- | | | | | |
| | ethyl)]aminopiperidine | | | | | |
| 21 | 1-(2-phenylethyl)-4-[N-(3- | Ph | CH₂CH₂ | CH₂CH₂CH₂ | Ph | CH₂CH₂Ph |
| | phenylpropyl)-N-phenyl- | | | | | |
| | ethyl]aminopiperidine | | | | | |
| 22 | 1-(2-phenylethyl)-4-[N- | Ph | CH₂CH₂ | CH₂CH₂CH₂ | Ph | CH₂Ph |
| | benzyl-N-(3-phenylpropyl)]- | | | | | |
| | aminopiperidine | | | | | |
| 23 | 1-(2-phenylethyl)-4-[N- | Ph | CH₂CH₂ | CH₂CH₂CH₂ | Ph | Et |
| | ethyl-N-(3-phenylpropyl)]- | | | | | |
| | aminopiperidine | | | | | |
| 24 | 1-(2-phenylethyl)-4-[N- | Ph | CH₂CH₂ | CH₂CH₂CH₂ | Ph | Pr |
| | propyl-N-(3-phenylpropyl)]- | | | | | |
| | aminopiperidine | | | | | |
| 25 | 1-(2-phenylethyl)-4-[N- | Ph | CH₂CH₂ | CH₂CH₂CH₂ | Ph | n-Bu |
| | butyl-N-3-(3-phenyl- | | | | | |
| | propyl)]aminopiperidine | | | | | |
| 26 | 1-(2-phenylethyl)-4-[N-2- | Ph | CH₂CH₂ | CH₂CH₂CH₂ | Ph | CH₂CH₂Ph |
| | (3,4-dimethoxy)phenyl- | | | | | [(3,4)-Ome-] |
| | ethyl-N-(3-phenylpropyl)]- | | | | | |
| | aminopiperidine | | | | | |
| 27 | 1-(2-phenylethyl)-4-[N-2- | Ph | CH₂CH₂ | CH₂CH₂CH₂ | Ph | (CH₂)₂N(Me)₂ |
| | dimethylaminoethyl-N-(3- | | | | | |
| | phenylpropyl)]amino- | | | | | |
| | piperidine | | | | | |
| 28 | 1-(2-phenylethyl)-4-[N-2- | Ph | CH₂CH₂ | CH₂CH₂CH₂ | Ph | CH₂CH₂OCH₃ |
| | methoxyethyl-N-(3-phenyl- | | | | | |
| | propyl)]aminopiperidine | | | | | |

Among the preferred compounds of formula (II), mention may be made of those mentioned above except for compound 6.

Among the preferred compounds of formula (III), mention may be made of compounds 1, 7 to 11 and 14 to 28 mentioned above.

Compounds (I) that are particularly preferred are compounds 1, 5, 6, 21, 25 and 27, and especially 1 and 5.

Compounds (II) that are particularly preferred are compounds 1, 5, 21, 25 and 27, and especially 1 and 5.

Compounds (III) that are particularly preferred are compounds 1, 21, 25 and 27, and especially 1.

In general, the compounds of formula (I) for which R is a hydrogen atom may be prepared according to Schemes I and II below by alkylation of 1,1-dimethyl-4-oxo-piperidinium iodide with an alkylamine(III), especially at a temperature of between 40° C. and 100° C., in particular in the presence of a mineral base (for example sodium bicarbonate or sodium hydroxide) in a water/ethanol mixture to form a piperidone (A), which is isolated and then purified, for example by chromatography on silica gel.

The piperidone (A) obtained is then alkylated and reduced with an alkylamine (B), especially in the presence of acetic acid (1 molar equivalent) and of NaBH(OAc)₃ (sodium triacetoxyborohydride) (2 molar equivalents), in particular in dichloromethane, and especially at room temperature (25° C.).

The reaction medium is then extracted 3 times with water at acidic pH. The aqueous phases are then combined and extracted 3 times with dichloromethane. The organic phases are combined, dried and concentrated to obtain the expected product (Ia), this product optionally being purified on silica gel and/or by precipitation.

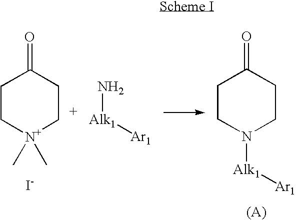

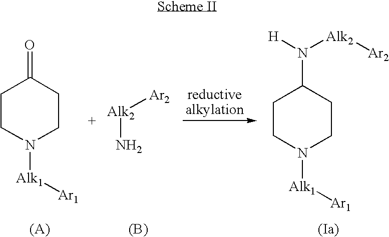

Compounds 1 to 16 were synthesized according to the general process of Scheme II.

The compounds of formula (I) for which R is other than a hydrogen atom may be prepared according to Scheme III described below.

Compound (Ia) is acylated with the acid chloride of formula RCOCl in the presence of an organic base (for example pyridine or triethylamine), to give an intermediate amide, which is reduced in the presence of LiAlH₄ (lithium aluminium double hydride), especially in an ether solvent (for example dioxane, diethyl ether or tetrahydrofuran), especially by heating to a temperature of between 40° C. and 100° C., and thus makes it possible to obtain compound (Ib).

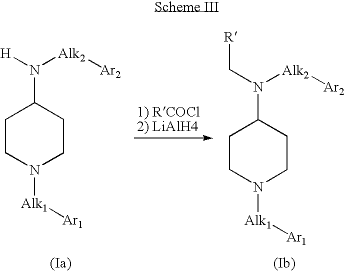

Compounds 17 to 28 were synthesized according to this general process of Scheme III.

The amount of 4-aminopiperidine compound of formula (I), (II) or (III) that may be used according to the invention is not limited and depends on the desired effect, and may thus vary within a wide range.

To give an order of magnitude, the compounds of formula (I), (II) or (III) may be used in an amount representing from 0.01% to 10% of the total weight of the composition, preferentially in an amount representing from 0.05% to 5% of the total weight of the composition and more preferentially in an amount representing from 0.1% to 2% of the total weight of the composition.

The composition according to the invention is preferably suitable for topical application to the skin and thus preferably contains a physiologically acceptable medium, i.e. a medium that is compatible with the skin and, where appropriate, with its integuments (eyelashes, nails or hair) and/or mucous membranes. This medium is advantageously cosmetically acceptable, i.e. it does not cause any itching, stinging or redness liable to put the user off using the composition, and it has a pleasant appearance, odour and feel.

This composition may be in any presentation form, including any form normally used in cosmetics, and it may especially be in the form of an optionally gelled solution, a dispersion of the lotion type, optionally a two-phase lotion, an emulsion obtained by dispersing a fatty phase in an aqueous phase (O/W emulsion) or conversely (W/O emulsion), or a triple emulsion (W/O/W or O/W/O emulsion) or a vesicular dispersion of ionic and/or nonionic type. These compositions are prepared according to the usual methods. A composition in the form of an oil-in-water emulsion is preferably used according to this invention.

This composition may be more or less fluid and may have the appearance of a white or coloured cream, an ointment, a milk, a lotion, a serum, a paste or a mousse. It may optionally be applied in the form of an aerosol. It may also be in solid form, in particular in the form of a stick. It may be used as a care product and/or as a makeup product for the skin.

The composition used according to the invention may also contain adjuvants, including those that are common in cosmetics, such as oils, waxes, emulsifiers, gelling agents, film-forming polymers, preserving agents, fragrances, fillers, UV-screening agents, bactericides, odour absorbers, dyestuffs, hydrophilic or lipophilic active agents, plant extracts and antioxidants. The amounts of these various adjuvants include those conventionally used in the field under consideration, and, for example, from 0.01% to 20% relative to the total weight of the composition. Depending on their nature, these adjuvants may be introduced into the fatty phase, into the aqueous phase, or into lipid vesicles. In any case, these adjuvants, and also the proportions thereof, will be chosen so as not to harm the desired properties of the compounds according to the invention.

When the composition used according to the invention is an emulsion, the proportion of the fatty phase may range for example from 5% to 80% by weight and preferably from 5% to 50% by weight relative to the total weight of the composition. The oils, emulsifiers and co-emulsifiers used in the composition in emulsion form are chosen from those conventionally used in the field under consideration. The emulsifier and co-emulsifier are typically present in the composition in a proportion ranging from 0.3% to 30% by weight and preferably from 0.5% to 20% by weight relative to the total weight of the composition.

Oils that may be used in the invention include mineral oils (liquid petroleum jelly), oils of plant origin (avocado oil or soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids and waxes (carnauba wax or ozokerite) may also be used as fatty substances.

As examples of emulsifiers and co-emulsifiers that may be used in the invention, mention may be made of fatty acid esters of polyethylene glycol such as PEG-100 stearate, and fatty acid esters of glycerol such as glyceryl stearate.

Hydrophilic gelling agents/thickeners that may be mentioned in particular include carboxyvinyl polymers (carbomer), acrylic copolymers such as acrylate/alkylacrylate copolymers, polyacrylamides, polysaccharides, natural gums and clays, and lipophilic gelling agents/thickeners that may be mentioned include modified clays, for instance bentones, metal salts of fatty acids and hydrophobic silica.

As active agents, it will be advantageous to introduce into the composition used according to the invention at least one compound chosen from: desquamating agents; moisturizers; depigmenting or propigmenting agents; antiglycation agents; NO-synthase inhibitors; agents for stimulating the synthesis of dermal or epidermal macromolecules and/or for preventing their degradation; agents for stimulating the proliferation of fibroblasts and/or keratinocytes or for stimulating the differentiation of keratinocytes; other muscle-relaxing agents and/or dermo-decontracting agents; tensioning agents; antipollution agents and/or free-radical scavengers; agents acting on the capillary circulation; agents acting on the energy metabolism of cells; and mixtures thereof.

Examples of such additional compounds include: retinol and its compounds such as retinyl palmitate; ascorbic acid and its compounds such as magnesium ascorbyl phosphate and ascorbyl glucoside; tocopherol and its compounds such as tocopheryl acetate; nicotinic acid and its precursors such as nicotinamide; ubiquinone; glutathione and its precursors such as L-2-oxothiazolidine-4-carboxylic acid; plant extracts and especially plant proteins and hydrolysates thereof, and also plant hormones; marine extracts such as algal extracts; bacterial extracts; sapogenins such as diosgenin and extracts of wild yam containing diosgenin; ceramides; hydroxy acids such as salicylic acid and 5-n-octanoylsalicylic acid; resveratrol; oligopeptides and pseudodipeptides and acyl compounds thereof; manganese and magnesium salts, in particular the gluconates; and mixtures thereof.

As mentioned previously, the composition according to the invention may also contain UVA-active and/or UVB-active photoprotective agents, in the form of organic or mineral compounds, the latter optionally being coated to make them hydrophobic.

The organic photoprotective agents may be chosen especially from: anthranilates, in particular menthyl anthranilate; benzophenones, in particular benzophenone-1, benzophenone-3, benzophenone-5, benzophenone-6, benzophenone-8, benzophenone-9, benzophenone-12 and, preferentially, benzophenone-3(oxybenzone) or benzophenone-4 (Uvinul MS40 available from BASF); benzylidenecamphors, in particular 3-benzylidenecamphor, benzylidenecamphorsulfonic acid, camphor benzalkonium methosulfate, polyacrylamidomethylbenzylidenecamphor, terephthalylidenedicamphorsulfonic acid and, preferentially, 4-methylbenzylidenecamphor (Eusolex 6300 available from Merck); benzimidazoles, in particular benzimidazilate (Neo Heliopan AP available from Haarmann & Reimer), or phenylbenzimidazolesulfonic acid (Eusolex 232 available from Merck); benzotriazoles, in particular drometrizole trisiloxane, or methylenebis-benzotriazolyltetramethylbutylphenol (Tinosorb M available from Ciba); cinnamates, in particular cinoxate, DEA methoxycinnamate, diisopropyl methylcinnamate, glyceryl ethylhexanoate dimethoxycinnamate, isopropyl methoxycinnamate, isoamyl cinnamate and, preferentially, ethocrylene (Uvinul N35 available from BASF), octyl methoxycinnamate (Parsol MCX available from Hoffmann La Roche), or octocrylene (Uvinul 539 available from BASF); dibenzoylmethanes, in particular butylmethoxydibenzoylmethane (Parsol 1789); imidazolines, in particular ethylhexyl dimethoxy-benzylidene dioxoimidazoline; PABAs, in particular ethyl dihydroxypropyl PABA, ethylhexyldimethyl PABA, glyceryl PABA, PABA, PEG-25 PABA and, preferentially, diethylhexylbutamidotriazone (Uvasorb HEB available from 3V Sigma), ethylhexyltriazone (Uvinul T150 available from BASF) or ethyl PABA (benzocaine); salicylates, in particular dipropylene glycol salicylate, ethylhexyl salicylate, homosalate or TEA salicylate; triazines, in particular anisotriazine (Tinosorb S available from Ciba); drometrizole trisiloxane.

The mineral photoprotective agents preferably consist of zinc oxide and/or titanium dioxide, preferably of nanometric size, optionally coated with alumina and/or stearic acid.

The composition according to the invention is advantageously intended to be applied to the areas of the face and/or the forehead marked by expression wrinkles, and/or on individuals with expression wrinkles.

The wrinkles concerned are preferably those lying radially around the mouth and/or the eyes, in particular crow's-feet wrinkles, and/or lying on the forehead, in particular the “lion” wrinkles located in the glabella or in the space between the eyebrows, and/or lying horizontally on the forehead.

The invention will now be illustrated by means of the non-limiting examples that follow. In these examples, the amounts are indicated as weight percentages.

### EXAMPLE 1

### Synthesis of 1-(2-phenylethyl)-4-[N-(3-phenylpropyl)]-aminopiperidine (compound 1)

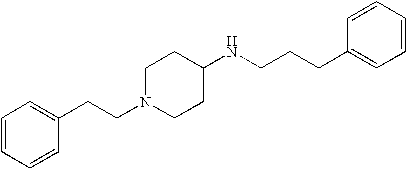

10 g of 1-phenylethyl-4-piperidone (1 molar equivalent) and 9.2 ml of 3-phenylpropylamine (1.3 molar equivalents) were placed in 100 ml of dichloromethane in the presence of 3.09 ml of acetic acid (1 molar equivalent) and 21.94 g of NaBH(OAc)₃ (2 molar equivalents), and the mixture was reacted at room temperature (25° C.) for 20 hours.

After treatment and purification, 15.8 g of product were obtained in the form of a brown oil (99% yield).

Mass spectrum in accordance with expected structure.

### EXAMPLE 2

### Synthesis of 1-(2-phenylethyl)-4-[N-ethyl-N-3-(phenylpropyl)]aminopiperidine (compound 23)

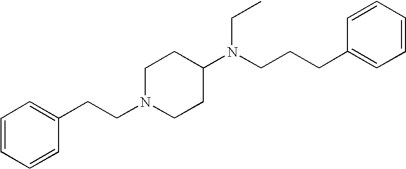

15.8 g of 1-(2-phenylethyl)-4-[N-(3-phenylpropyl)aminopiperidine (obtained according to Example 1) and 3.65 ml of acetyl chloride (1.05 molar equivalents) were placed in dichloromethane in the presence of 14.45 ml of triethylamine (2.1 molar equivalents), and the mixture was reacted at room temperature (25° C.) for 15 hours.

After work-up and purification on a sinter-funnel of silica, 15.5 g of 1-(2-phenylethyl)-4-N-[acetyl-N-3-(phenylpropyl)]aminopiperidine were obtained (87% yield).

Next, 15.5 g of the intermediate compound obtained were mixed with 8.5 g (5 molar equivalents) of LiAlH₄ in refluxing ethyl ether for 2 hours. After work-up and purification on a sinter-funnel of silica, 10.5 g of the expected product were obtained (70% yield).

### EXAMPLE 3

### Demonstration of the Dermo-Decontracting Effect of the 4-aminopiperidine Compounds According to the Invention

a) Principle of the Test

The principle of this test consists in studying the effect of the test product on a model of equivalent dermis consisting of a collagen matrix seeded with normal human fibroblasts.

These conditions are intended to mimic in vitro the dermal contractile phenomena that take place during facial movements. The reason for this is that, under these conditions, the cells spontaneously exert tensile forces that induce a shrinkage of the collagen gel. This results in a decrease in the total surface area of the equivalent dermis over time. Measurement of this area makes it possible to evaluate the relaxation effects of the substances placed in contact beforehand with the equivalent dermis.

b) Protocol

Two series of attached equivalent dermides containing normal human fibroblasts are prepared: a control series without any treatment, and a series treated with the test compound (1 μM). The experiment is repeated three times.

The dermal equivalents are prepared as described in Asselineau et al., Exp. Cell. Res., 1985, 159, 536-539; Models in dermatology, 1987, vol. 3, pp. 1-7, in the following proportions:

| | |
|---|---|
| MEM medium (1.76×) with or without compound | 45% |
| Foetal calf serum: | 10% |
| NaOH (0.1N): | 5% |
| Acetic acid (1/1000): | 4% |
| Collagen: | 26% |
| Fibroblasts: | 11% |

The treated equivalent dermis differs from the control equivalent dermis in that 1 μM of the test compound has been added thereto.

The collagen used is type I collagen (commercial solution). It is extracted from rat tails or from calf skin by acid hydrolysis and stored in acidic medium at +4° C.; it polymerizes naturally by heating to 37° C. and by reducing the acidity level. The collagen is predialysed against successive baths of water+acetic acid.

The protocol is as follows: 1.76×MEM medium is introduced into a 50 ml centrifuge tube stored, in crushed ice, in the presence of additives (1% glutamine, 1% non-essential amino acids, 1% sodium pyruvate, 1% fungizone and 1% penicillin/streptomycin), foetal calf serum, 0.1N sodium hydroxide NaOH. Fibroblasts isolated from human skin explants are then added at a concentration of 1.5×10⁵ cells per 1 ml of culture medium.

A 1/1000 volume/volume mixture of collagen in acetic acid is then added slowly, down the wall of the tube so as to observe the appearance of a whitish cloud.

The assembly is then mixed cautiously and distributed into the wells of a 12-well culture plate (such as Costar reference 3512), at a rate of 2 ml of mixture per well. The final cell concentration is 3×10⁴ cells/equivalent dermis, with a final collagen concentration of 1 mg/ml. The culture plate is then placed in an incubator at 37° C. with 5% CO₂.

Once formed after polymerization of the collagen, the equivalent dermides are left adhering to the culture support for 3 days and then detached from the support so that the contraction can start. These attached equivalent dermides are removed from the incubator in order to take images so as to measure their surface area, this being done for each point of the contraction kinetics (0, 4, 8 and 24 hours). They are immediately returned to the incubator between each measuring point.

The evaluation of the spontaneous contraction of the equivalent dermides that have been treated (with the test compound) and of the control equivalent dermides (without test compound) is performed by measuring their surface area, at different times after the start of the spontaneous contraction.

To do this, a digital image is acquired for each treated or untreated equivalent dermis using a camera (CCD camera—Iris Sony DXC-107P) and the surface area is then calculated on each image by means of an image analysis system (Zeiss Axiovision 3.0). This surface area measurement has a corresponding percentage of contraction equal to the ratio of the surface areas according to the formula:
% contraction=(Sw−Si)/Sw×100
in which:
“Sw” represents the surface area of a culture plate well; it corresponds to the total surface area of the equivalent dermis before contraction
“Si” represents the surface area of the equivalent dermis at the instant i of the contraction kinetics.
   c) Results

1-(2-Phenylethyl)-4-[N-(3-phenylpropyl)]aminopiperidine (compound 1) of formula 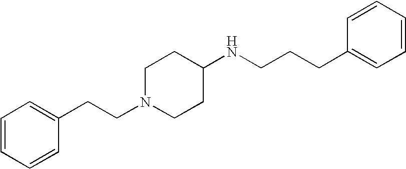
reduces the contraction of fibroblasts by 21% on average over the duration of the experiment (tested at 1 μM), compared with the control.

1-(2-Phenylethyl)-4-[N-(1-phenylethyl)]aminopiperidine (compound 5) of formula: 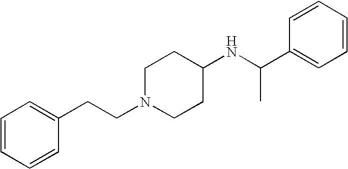
reduces the contraction of fibroblasts by 40% on average over the duration of the experiment (tested at 10 μM), compared with the control.

The two test compounds thus have a significant dermo-decontracting effect.

### EXAMPLE 4

A skin care cream having the composition below (weight percentage) is prepared:

| | |
|---|---|
| Compound of Example 1 | 0.10% |
| Stearic acid | 3.00% |
| Mixture of glyceryl monostearate and | 2.50% |
| polyethylene glycol stearate (100 EO) | |
| Polyethylene glycol stearate (20 EO) | 1.00% |
| Cyclopentadimethylsiloxane | 10.00% |
| Fillers | 3.00% |
| Plant oils | 7.00% |
| Synthetic oils | 6.00% |
| Preserving agents | 1.20% |
| Oxyethylenated dimethylsiloxane (16 EO) | 1.00% |
| containing methoxy end groups | |
| Silicone gum | 0.20% |
| Acrylic copolymer as an inverse emulsion | 1.70% |
| (Simulgel 600 from SEPPIC) | |
| Stearyl alcohol | 1.00% |
| Water | qs 100% |

This cream is intended to be applied to the face and the forehead to relax the features and to decontract facial skin.

In the composition described above, compound 1 may be replaced with compound 5 or compound 23.

The above written description of the invention provides a manner and process of making and using it such that any person skilled in this art is enabled to make and use the same, this enablement being provided in particular for the subject matter of the appended claims, which make up a part of the original description and including the use of at least one 4-aminopiperidine compound of formula (I): 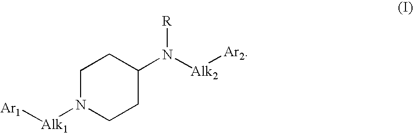
in which:
Alk₁ and Alk₂ denote, independently of each other, a linear saturated C₁-C₁₀ or unsaturated C₂-C₁₀ or branched saturated or unsaturated C₃-C₁₀ alkylene radical;
Ar₁ denotes a phenyl group optionally substituted with one or more radicals, which may be identical or different, chosen from —F, —CF₃, —R₁, —OR₁, —NR₁R₂;
Ar₂ denotes a phenyl group optionally substituted with one or more radicals, which may be identical or different, chosen from —F, —CF₃, —NR₁R₂;
R denotes a hydrogen atom or a saturated or unsaturated, linear C₁-C₁₀ or branched C₃-C₁₀ alkyl radical, optionally substituted with a group chosen from —Ar₁, —OR₁, —NR₁R₂;
   R₁ and R₂ denoting, independently of each other, a saturated, linear C₁-C₇, or unsaturated C₂-C₇ or saturated or unsaturated branched or cyclic C₃-C₇ alkyl radical;
   and the salts, optical isomers and solvates thereof,
   as an agent for combating wrinkles, especially expression wrinkles, and/or for decontracting the skin and/or relaxing the features.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

The invention method and composition is preferably used by subjects desirous of the benefits noted herein, subjects “in need of” these benefits. Such subjects are typically suffering from wrinkles, especially expression wrinkles, and/or are in need of decontracting the skin and/or relaxing the features, such as by self diagnosis or cosmetician or medical diagnosis, or are at recognized and appreciated risk of developing such conditions and who use the invention methods and compositions to combat these effects. In this regard, the invention process can be viewed as one for delaying the onset of the appearance of, and/or for reducing signs of, wrinkles, especially expression wrinkles.

Naturally, one using the invention as disclosed will use an amount of the invention composition effective to reduce the signs of ageing. Such amount is inclusive of an amount of the compositions described herein at the disclosed concentrations of active ingredients sufficient to cover the area of the skin being treated in a single application, and of course includes that amount applied upon repeated application, for example on a daily basis over a course of days, weeks, etc. In a preferred embodiment the invention process includes multiple applications of the invention composition to the area(s) of skin in need of attention.

In the claims that follow, a claim set forth such as:
A composition according to [a previous claim], wherein:
   R denotes a hydrogen atom, and
   Alk₁ denotes a saturated, linear C₂-C₁₀ or branched C₃-C₁₀ alkylene radical; and
   Alk₂ denotes a saturated, linear C₁-C₁₀ or branched C₃-C₁₀ alkylene radical; or
   R denotes a saturated, linear C₁-C₄ or branched C₃-C₄ alkyl radical, optionally substituted with a group chosen from —Ar₁, —OR₁, —NR₁R₂; and
   Alk₁ and Alk₂ denote, independently of each other, a saturated, linear C₁-C₁₀ or branched C₃-C₁₀ alkylene radical;
   Ar₁ denotes a phenyl group optionally substituted with one or more radicals, which may be identical or different, chosen from —R₁, —OR₁;
   Ar₂ denotes a phenyl group optionally substituted with one or more —CF₃ radicals;
R₁ and R₂ denoting, independently of each other, a saturated, linear C₁-C₄ alkyl radical
   provides alternative groupings for R, Alk₁ and Alk₂, and defines Ar₁, Ar₂, R₁ and R₂ regardless which grouping for R, Alk₁ and Alk₂ is chosen.

1. A method for combating wrinkles and/or for decontracting the skin and/or relaxing the features, comprising applying at least one 4-aminopiperidine compound of formula (I): 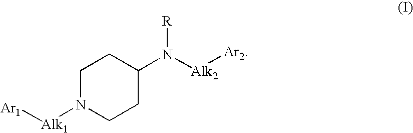
in which:
Alk₁ and Alk₂ denote, independently of each other, a linear saturated C₁-C₁₀ or unsaturated C₂-C₁₀ or branched saturated or unsaturated C₃-C₁₀ alkylene radical;
Ar₁ denotes a phenyl group optionally substituted with one or more radicals, which may be identical or different, chosen from —F, —CF₃, —R₁, —OR₁, —NR₁R₂;
Ar₂ denotes a phenyl group optionally substituted with one or more radicals, which may be identical or different, chosen from —F, —CF₃, —NR₁R₂;
R denotes a hydrogen atom or a saturated or unsaturated, linear C₁-C₁₀ or branched C₃-C₁₀ alkyl radical, optionally substituted with a group chosen from —Ar₁, —OR₁, —NR₁R₂;
R₁ and R₂ denoting, independently of each other, a saturated, linear C₁-C₇, or unsaturated C₂-C₇ or saturated or unsaturated branched or cyclic C₃-C₇ alkyl radical;
and the salts, optical isomers and solvates thereof,
to skin in need thereof.

2. The method according to claim 1, wherein:
Alk₁ and Alk₂ denote, independently of each other, a saturated, linear C₁-C₁₀ or branched C₃-C₁₀ alkylene radical;
Ar₁ denotes a phenyl group optionally substituted with one or more radicals, which may be identical or different, chosen from —R₁, —OR₁;
Ar₂ denotes a phenyl group optionally substituted with one or more radicals —CF₃;
R denotes a hydrogen atom or a saturated, linear C₁-C₄ or branched C₃-C₄ alkyl radical, optionally substituted with a group chosen from —Ar₁, —OR₁, —NR₁R₂;
R₁ and R₂ denoting, independently of each other, a saturated, linear C₁-C₄ alkyl radical.

3. The method according to claim 1, wherein:
Alk₁ and Alk₂ denote, independently of each other, a saturated, linear C₁-C₄ or branched C₃-C₄ alkylene radical;
Ar₁ and Ar₂ denote a phenyl group;
R denotes a hydrogen atom or a saturated, linear C₁-C₄ or branched C₃-C₄ alkyl radical, optionally substituted with a group chosen from a phenyl radical, —NR₁R₂.

4. The method according to claim 1, wherein:
Alk₁ and Alk₂ denote, independently of each other, a saturated, linear C₁-C₄ or branched C₃-C₄ alkylene radical;
Ar₁ and Ar₂ denote a phenyl group;
R denotes a hydrogen atom.

5. The method according to claim 1, wherein said at least one 4-aminopiperidine compound of formula (I) comprises at least one of the following compounds:
1-(2-phenylethyl)-4-[N-(3-phenylpropyl)]aminopiperidine;
1-(2-phenylethyl)-4-(N-benzyl)aminopiperidine;
1-(2-phenylethyl)-4-[N-(3,5-bistrifluoromethyl)-benzyl]aminopiperidine;
1-(2-phenylethyl)-4-[N-(2-phenylethyl)]aminopiperidine;
1-(2-phenylethyl)-4-[N-(1-phenylethyl)]aminopiperidine;
1-benzyl-4-[N-(1-phenylethyl)]aminopiperidine;
1-[2-(3,4-dimethoxy)phenylethyl]-4-[N-(3-phenylpropyl)]aminopiperidine;
1-(3-phenylpropyl)-4-[N-(3-phenylpropyl)]aminopiperidine;
1-(2-phenylethyl)-4-[N-(4-phenylbutyl)]aminopiperidine;
1-(2-phenylethyl)-4-[N-(1-phenylpropyl)]aminopiperidine;
1-[2-(4-tert-butyl)phenylethyl]-4-[N-(3-phenylpropyl)]aminopiperidine;
1-(2-phenylethyl)-4-[N-(1S)-1-phenylethyl]aminopiperidine;
1-[2-(4-tert-butyl)phenylethyl]-4-[N-(1-phenylethyl)]aminopiperidine;
1-(4-phenylbutyl)-4-[N-(3-phenylpropyl)]aminopiperidine;
1-(3-phenylpropyl)-4-[N-(1-phenylpropyl)]aminopiperidine;
1-(3-phenylpropyl)-4-[N-(1S)-1-phenylethyl]aminopiperidine;
1-(2-phenylethyl)-4-[N-methyl-N-(3-phenylpropyl)]-aminopiperidine;
1-(2-phenylethyl)-4-[N,N-di(2-phenylethyl)]aminopiperidine;
1-(2-phenylethyl)-4-[N-ethyl-N-(1-phenylethyl)]aminopiperidine;
1-(2-phenylethyl)-4-[N-(2-phenylpropyl)-N-(1-phenylethyl)]aminopiperidine;
1-(2-phenylethyl)-4-[N-(3-phenylpropyl)-N-phenylethyl]aminopiperidine;
1-(2-phenylethyl)-4-[N-benzyl-N-(3-phenylpropyl)]-aminopiperidine;
1-(2-phenylethyl)-4-[N-ethyl-N-(3-phenylpropyl)]-aminopiperidine;
1-(2-phenylethyl)-4-[N-propyl-N-(3-phenylpropyl)]-aminopiperidine;
1-(2-phenylethyl)-4-[N-butyl-N-3-(3-phenylpropyl)]-aminopiperidine;
1-(2-phenylethyl)-4-[N-2-(3,4-dimethoxy)phenylethyl-N-(3-phenylpropyl)]aminopiperidine;
1-(2-phenylethyl)-4-[N-2-dimethylaminoethyl-N-(3-phenylpropyl)]aminopiperidine; and
1-(2-phenylethyl)-4-[N-2-methoxyethyl-N-(3-phenylpropyl)]aminopiperidine.

6. The method according to claim 1, wherein said at least one 4-aminopiperidine compound of formula (I) is present in a composition comprising a physiologically acceptable medium.

7. A composition comprising, in a physiologically acceptable medium, at least one 4-aminopiperidine compound of formula (II): 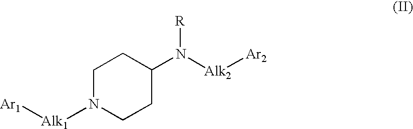
in which:
Alk₁ denotes a saturated or unsaturated, linear C₂-C₁₀ or branched C₃-C₁₀ alkylene radical;
Alk₂ denotes a saturated, linear C₁-C₁₀ or unsaturated C₂-C₁₀ or saturated or unsaturated branched C₃-C₁₀ alkylene radical;
Ar₁ denotes a phenyl group optionally substituted with one or more radicals, which may be identical or different, chosen from —F, —CF₃, —R₁, —OR₁, —NR₁R₂;
Ar₂ denotes a phenyl group optionally substituted with one or more radicals, which may be identical or different, chosen from —F, —CF₃, —NR₁R₂;
R denotes a hydrogen atom or a saturated or unsaturated, linear C₁-C₁₀ or branched C₃-C₁₀ alkyl radical, optionally substituted with a group chosen from —Ar₁, —OR₁, —NR₁R₂;
R₁ and R₂ denoting, independently of each other, a saturated, linear C₁-C₇, or unsaturated C₂-C₇ or saturated or unsaturated branched or cyclic C₃-C₇ alkyl radical;
and the salts, optical isomers and solvates thereof.

8. A composition according to claim 7, wherein:
R denotes a hydrogen atom, and
Alk₁ denotes a saturated, linear C₂-C₁₀ or branched C₃-C₁₀ alkylene radical; and
Alk₂ denotes a saturated, linear C₁-C₁₀ or branched C₃-C₁₀ alkylene radical; or
R denotes a saturated, linear C₁-C₄ or branched C₃-C₄ alkyl radical, optionally substituted with a group chosen from —Ar₁, —OR₁, —NR₁R₂; and
Alk₁ and Alk₂ denote, independently of each other, a saturated, linear C₁-C₁₀ or branched C₃-C₁₀ alkylene radical;
Ar₁ denotes a phenyl group optionally substituted with one or more radicals, which may be identical or different, chosen from —R₁, —OR₁;
Ar₂ denotes a phenyl group optionally substituted with one or more —CF₃ radicals;
R₁ and R₂ denoting, independently of each other, a saturated, linear C₁-C₄ alkyl radical.

9. A composition according to claim 7, wherein:
R denotes a hydrogen atom, and
Alk₁ denotes a saturated, linear C₂-C₄ or branched C₃-C₄ alkylene radical; and
Alk₂ denotes a saturated, linear C₁-C₄ or branched C₃-C₄ alkylene radical; or
R denotes a saturated, linear C₁-C₄ or branched C₃-C₄ alkyl radical, optionally substituted with a group chosen from —Ar₁, —OR₁, —NR₁R₂; and
Alk₁ and Alk₂ denote, independently of each other, a saturated, linear C₁-C₄ or branched C₃-C₄ alkylene radical;
Ar₁ and Ar₂ denote a phenyl group;
R₁ and R₂ denoting, independently of each other, a saturated, linear C₁-C₄ alkyl radical.

10. A composition according to claim 7, wherein:
R denotes a hydrogen atom, and
Alk₁ denotes a saturated, linear C₂-C₁₀ or branched C₃-C₁₀ alkylene radical; and
Alk₂ denotes a saturated, linear C₁-C₁₀ or branched C₃-C₁₀ alkylene radical; or
R denotes a saturated, linear C₁-C₄ or branched C₃-C₄ alkyl radical, optionally substituted with a group chosen from a phenyl radical, —NR₁R₂, and
Alk₁ and Alk₂ denote, independently of each other, a saturated, linear C₁-C₄ or branched C₃-C₄ alkylene radical;
Ar₁ and Ar₂ denote a phenyl group;
R₁ and R₂ denoting, independently of each other, a saturated, linear C₁-C₄ alkyl radical.

11. A composition according to claim 7, wherein:
R denotes a hydrogen atom,
Alk₁ denotes a saturated, linear C₂-C₁₀ or branched C₃-C₁₀ alkylene radical;
Alk₂ denotes a saturated, linear C₁-C₁₀ or branched C₃-C₁₀ alkylene radical;
Ar₁ and Ar₂ denote a phenyl group;
R₁ and R₂ denoting, independently of each other, a saturated, linear C₁-C₄ alkyl radical.

12. The composition according to claim 7, comprising at least one compound chosen from:
1-(2-phenylethyl)-4-[N-(3-phenylpropyl)]aminopiperidine;
1-(2-phenylethyl)-4-(N-benzyl)aminopiperidine;
1-(2-phenylethyl)-4-[N-(3,5-bistrifluoromethyl)-benzyl]aminopiperidine;
1-(2-phenylethyl)-4-[N-(2-phenylethyl)]aminopiperidine;
1-(2-phenylethyl)-4-[N-(1-phenylethyl)]aminopiperidine;
1-[2-(3,4-dimethoxy)phenylethyl]-4-[N-(3-phenylpropyl)]aminopiperidine;
1-(3-phenylpropyl)-4-[N-(3-phenylpropyl)]aminopiperidine;
1-(2-phenylethyl)-4-[N-(4-phenylbutyl)]aminopiperidine;
1-(2-phenylethyl)-4-[N-(1-phenylpropyl)]aminopiperidine;
1-[2-(4-tert-butyl)phenylethyl]-4-[N-(3-phenylpropyl)]aminopiperidine;
1-(2-phenylethyl)-4-[N-(1S)-1-phenylethyl]aminopiperidine;
1-[2-(4-tert-butyl)phenylethyl]-4-[N-(1-phenylethyl)]aminopiperidine;
1-(4-phenylbutyl)-4-[N-(3-phenylpropyl)]aminopiperidine;
1-(3-phenylpropyl)-4-[N-(1-phenylpropyl)]aminopiperidine;
1-(3-phenylpropyl)-4-[N-(1S)-1-phenylethyl]aminopiperidine;
1-(2-phenylethyl)-4-[N-methyl-N-(3-phenylpropyl)]-aminopiperidine;
1-(2-phenylethyl)-4-[N,N-di(2-phenylethyl)]aminopiperidine;
1-(2-phenylethyl)-4-[N-ethyl-N-(1-phenylethyl)]aminopiperidine;
1-(2-phenylethyl)-4-[N-(2-phenylpropyl)-N-(1-phenylethyl)]aminopiperidine;
1-(2-phenylethyl)-4-[N-(3-phenylpropyl)-N-phenylethyl]aminopiperidine;
1-(2-phenylethyl)-4-[N-benzyl-N-(3-phenylpropyl)]-aminopiperidine;
1-(2-phenylethyl)-4-[N-ethyl-N-(3-phenylpropyl)]-aminopiperidine;
1-(2-phenylethyl)-4-[N-propyl-N-(3-phenylpropyl)]-aminopiperidine;
1-(2-phenylethyl)-4-[N-butyl-N-3-(3-phenylpropyl)]-aminopiperidine;
1-(2-phenylethyl)-4-[N-2-(3,4-dimethoxy)phenylethyl-N-(3-phenylpropyl)]aminopiperidine;
1-(2-phenylethyl)-4-[N-2-dimethylaminoethyl-N-(3-phenylpropyl)]aminopiperidine; and
1-(2-phenylethyl)-4-[N-2-methoxyethyl-N-(3-phenylpropyl)]aminopiperidine.

13. The composition according to claim 7, wherein said composition is a cosmetic or dermatological composition.

14. The composition according to claim 7, further comprising at lest one ingredient chosen from oils, waxes, emulsifiers, gelling agents, film-forming polymers, preserving agents, fragrances, fillers, UV-screening agents, bactericides, odour absorbers, dyestuffs, hydrophilic or lipophilic active agents, plant extracts and antioxidants.

15. A 4-Aminopiperidine compound of formula (III): 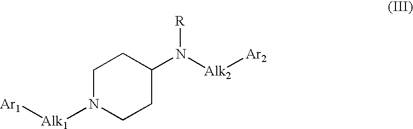
in which:
Alk₁ denotes a saturated or unsaturated, linear C₂-C₁₀ or branched C₃-C₁₀ alkylene radical;
Alk₂ denotes a saturated or unsaturated, linear or branched C₃-C₁₀ alkylene radical;
Ar₁ denotes a phenyl group optionally substituted with one or more radicals, which may be identical or different, chosen from —F, —CF₃, —R₁, —OR₁, —NR₁R₂;
Ar₂ denotes a phenyl group optionally substituted with one or more radicals, which may be identical or different, chosen from —F, —CF₃, —NR₁R₂;
R denotes a hydrogen atom or a saturated or unsaturated, linear C₁-C₁₀ or branched C₃-C₁₀ alkyl radical, optionally substituted with a group chosen from —Ar₁, —OR₁, —NR₁R₂;
R₁ and R₂ denoting, independently of each other, a saturated or unsaturated, linear C₁-C₇ or branched or cyclic C₃-C₇ alkyl radical;
and the salts, optical isomers and solvates thereof.

16. A compound according to claim 15, wherein:
R denotes a hydrogen atom, and
Alk₁ denotes a saturated, linear C₂-C₄ or branched C₃-C₄ alkylene radical; and
Alk₂ denotes a saturated, linear or branched C₃-C₄ alkylene radical; or
R denotes a saturated, linear C₁-C₄ or branched C₃-C₄ alkyl radical, optionally substituted with a group chosen from —Ar₁, —OR₁, —NR₁R₂; and
Alk₁ and Alk₂ denote, independently of each other, a saturated, linear C₁-C₄ or branched C₃-C₄ alkylene radical;
Ar₁ and Ar₂ denote a phenyl group;
R₁ and R₂ denoting, independently of each other, a saturated, linear C₁-C₄ alkyl radical.

17. A compound according to claim 15, wherein:
Alk₁ denotes a saturated, linear C₂-C₄ or branched C₃-C₄ alkylene radical;
Alk₂ denotes a saturated, linear or branched C₃-C₄ alkylene radical;
Ar₁ and Ar₂ denote a phenyl group;
R denotes a hydrogen atom or a saturated, linear C₁-C₄ or branched C₃-C₄ alkyl radical, optionally substituted with a group chosen from —Ar₁, —OR₁, —NR₁R₂;
R₁ and R₂ denoting, independently of each other, a saturated, linear C₁-C₄ alkyl radical.

18. A compound according to claim 15, wherein:
R denotes a hydrogen atom, and
Alk₁ denotes a saturated, linear C₂-C₄ or branched C₃-C₄ alkylene radical; and
Alk₂ denotes a saturated, linear or branched C₃-C₄ alkylene radical; or
R denotes a saturated, linear C₁-C₄ or branched C₃-C₄ alkyl radical, optionally substituted with a group chosen from a phenyl radical, —NR₁R₂, and
Alk₁ and Alk₂ denote, independently of each other, a saturated, linear C₁-C₄ or branched C₃-C₄ alkylene radical;
Ar₁ and Ar₂ denote a phenyl group;
R₁ and R₂ denoting, independently of each other, a saturated, linear C₁-C₄ alkyl radical.

19. A compound according to claim 15, wherein:
R denotes a hydrogen atom, and
Alk₁ denotes a saturated, linear C₂-C₄ or branched C₃-C₄ alkylene radical; and
Alk₂ denotes a saturated, linear or branched C₃-C₄ alkylene radical;
Ar₁ and Ar₂ denote a phenyl group;
R₁ and R₂ denoting, independently of each other, a saturated, linear C₁-C₄ alkyl radical.

20. A compounds according to claim 15, chosen from:
1-(2-phenylethyl)-4-[N-(3-phenylpropyl)]aminopiperidine;
1-[2-(3,4-dimethoxy)phenylethyl]-4-[N-(3-phenylpropyl)]aminopiperidine;
1-(3-phenylpropyl)-4-[N-(3-phenylpropyl)]aminopiperidine;
1-(2-phenylethyl)-4-[N-(4-phenylbutyl)]aminopiperidine;
1-(2-phenylethyl)-4-[N-(1-phenylpropyl)]aminopiperidine;
1-[2-(4-tert-butyl)phenylethyl]-4-[N-(3-phenylpropyl)]aminopiperidine;
1-(4-phenylbutyl)-4-[N-(3-phenylpropyl)]aminopiperidine;
1-(3-phenylpropyl)-4-[N-(1-phenylpropyl)]aminopiperidine;
1-(3-phenylpropyl)-4-[N-(1S)-1-phenylethyl]aminopiperidine;
1-(2-phenylethyl)-4-[N-methyl-N-(3-phenylpropyl)]-aminopiperidine;
1-(2-phenylethyl)-4-[N,N-di(2-phenylethyl)]aminopiperidine;
1-(2-phenylethyl)-4-[N-ethyl-N-(1-phenylethyl)]aminopiperidine;
1-(2-phenylethyl)-4-[N-(2-phenylpropyl)-N-(1-phenylethyl)]aminopiperidine;
1-(2-phenylethyl)-4-[N-(3-phenylpropyl)-N-phenylethyl]aminopiperidine;
1-(2-phenylethyl)-4-[N-benzyl-N-(3-phenylpropyl)]-aminopiperidine;
1-(2-phenylethyl)-4-[N-ethyl-N-(3-phenylpropyl)]-aminopiperidine;
1-(2-phenylethyl)-4-[N-propyl-N-(3-phenylpropyl)]-aminopiperidine;
1-(2-phenylethyl)-4-[N-butyl-N-3-(3-phenylpropyl)]-aminopiperidine;
1-(2-phenylethyl)-4-[N-2-(3,4-dimethoxy)phenylethyl-N-(3-phenylpropyl)]aminopiperidine;
1-(2-phenylethyl)-4-[N-2-dimethylaminoethyl-N-(3-phenylpropyl)]aminopiperidine; and
1-(2-phenylethyl)-4-[N-2-methoxyethyl-N-(3-phenylpropyl)]aminopiperidine.

21. A process for preparing a compound of formula (Ia) comprising the alkylation of 1,1-dimethyl-4-oxopiperidinium iodide with an alkylamine to form a piperidone (A), the piperidone (A) being alkylated and reduced with an alkylamine (B) to give compound (Ia): 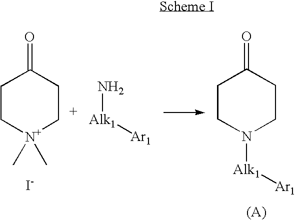 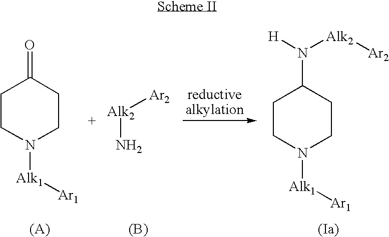
in which:
Alk₁ denotes a saturated or unsaturated, linear C₂-C₁₀ or branched C₃-C₁₀ alkylene radical;
Alk₂ denotes a saturated or unsaturated, linear or branched C₃-C₁₀ alkylene radical;
Ar₁ denotes a phenyl group optionally substituted with one or more radicals, which may be identical or different, chosen from —F, —CF₃, —R₁, —OR₁, —NR₁R₂;
Ar₂ denotes a phenyl group optionally substituted with one or more radicals, which may be identical or different, chosen from —F, —CF₃, —NR₁R₂; and
R₁ and R₂ denoting, independently of each other, a saturated or unsaturated, linear C₁-C₇ or branched or cyclic C₃-C₇ alkyl radical; or
a process for preparing a compound of formula (Ib) comprising acylating compound (Ia) with an acid chloride of formula RCOCl in the presence of an organic base to give an intermediate amide, which is reduced in the presence of LiAlH₄ to give compound (Ib): 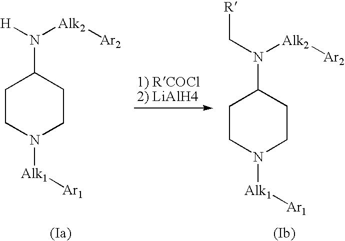
wherein:
Alk₁ denotes a saturated or unsaturated, linear C₂-C₁₀ or branched C₃-C₁₀ alkylene radical;
Alk₂ denotes a saturated or unsaturated, linear or branched C₃-C₁₀ alkylene radical;
Ar₁ denotes a phenyl group optionally substituted with one or more radicals, which may be identical or different, chosen from —F, —CF₃, —R₁, —OR₁, —NR₁R₂;
Ar₂ denotes a phenyl group optionally substituted with one or more radicals, which may be identical or different, chosen from —F, —CF₃, —NR₁R₂;
R′ denotes a saturated or unsaturated, linear C₁-C₁₀ or branched C₃-C₁₀ alkyl radical, optionally substituted with a group chosen from —Ar₁, —OR₁, —NR₁R₂;
R₁ and R₂ denoting, independently of each other, a saturated or unsaturated, linear C₁-C₇ or branched or cyclic C₃-C₇ alkyl radical.
